(19) United States
(12) Patent Application Publication
(10) Pub. No.: US 2006/0073502 A1
(43) Pub. Date: Apr. 6, 2006

(54) Apparatus for detecting bio-bonding and method thereof

(76) Inventors: Sung-hee Lee, Suwon-si (KR); Jung-ho Kang, Suwon-si (KR); Young-il Kim, Suwon-si (KR); Moon-chul Lee, Suwon-si (KR); Tae-sik Park, Suwon-si (KR)

Correspondence Address: CANTOR COLBURN, LLP, 55 GRIFFIN ROAD SOUTH, BLOOMFIELD, CT, 06002, US

(21) Appl. No.: 11/225,507

(22) Filed: Sep. 13, 2005

(30) Foreign Application Priority Data
Sep. 13, 2004 (KR) 2004-72863

(51) Int. Cl. C12Q 1/68; C12M 1/34

(52) U.S. Cl. 435/6; 435/287.2

(57) ABSTRACT

An apparatus and method for detecting a bio-bonding includes monitoring of a change of an electrical property before and after bio-bonding. The apparatus includes a signal transducer section having a substrate, a metallic thin film stepped on a surface of a certain region of the substrate, probe biomolecules formed on the metallic thin film, an input port connected with one side of the signal transducer section and into which a signal for monitoring an electrical property of the signal transducer section is input and a detector section for characteristic quantity connected with the other side of the signal transducer section so as to monitor an electrical property of the signal transducer section. The probe biomolecules are immobilized on the metallic thin film. The bio-bonding is detected through monitoring the electrical property of the signal transducer section before and after the bio-bonding.

This application claims priority to Korean Patent Application No. 2004-72863 filed on Sep. 13, 2004, and all the benefits accruing therefrom under 35 U.S.C. §119, the contents of which are herein incorporated by reference in its entirety.

### BACKGROUND OF THE INVENTION

1. Field of the Invention

The present invention generally relates to an apparatus for detecting a bio-bonding and a method thereof. More particularly, the present invention relates to an apparatus for detecting a bio-bonding and a method thereof, in which the bio-bonding is detected through the monitoring of a change of an electrical property before and after the bio-bonding.

2. Description of the Related Art

A bio chip is a biological micro chip that hundreds to hundreds of thousands of biomolecules, such as analyzed sequences of DNA, DNA segments and RNA, are arranged at intervals and attached on a small solid board. The silicon board may be made of glass, silicon or nylon, or the like, such that the expression methods, the distribution aspects, the mutation and like characteristics of genes can be analyzed. On the surface of the bio chip are fixed materials functioning as a probe that searches particular genetic information included in a sample. By reacting the sample to be analyzed with the bio chip, materials included in the sample are reacted with a probe fixed to the surface of the bio chip. These materials are respectively bonded to the probe to form a state of hybridization. To detect and interpret the bonding makes it possible to obtain information on the materials included in the sample.

Technologies concerned with the bio chip include a probe attaching and fixing technique, a signal detecting technique and an information processing technique. The signal detecting method may include a laser-induced fluorescence detecting method, an electrochemical detecting method, a mass detecting method and a mechanical detecting method.

FIGS. 1A to 1E illustrate bio-bonding detecting apparatus and methods of the prior art.

FIG. 1A is a view illustrating a laser-induced fluorescence detecting method. The laser-induced fluorescence detecting method includes binding a fluorescent material to a sample. The sample and probe biomolecules are reacted. A result of the reaction is checked with a fluorescence detector to optically determine whether the probe is bonded. This method, however, requires a pre-processing reaction for binding the fluorescent material to the sample before the bonding reaction between the probe and the sample, possibly causing a loss or a contamination of the sample. Other disadvantages includes an optical reader system for reading the result after the bonding reaction between the probe biomolecules and the sample is complicated, a high-priced reader is required, the optical detecting method makes it difficult to provide a compact sized optical detector from the optical detector, and a digitized output cannot be obtained.

FIG. 1B is the view of an exemplary mechanical detector for implementing the mechanical detecting method. The mechanical detecting method uses a micro-assembled cantilever for monitoring an intermolecular binding force before and after the bonding between the probe biomolecules and the sample. However, this method requires precise monitoring of the reflection of a cantilever beam. Additionally, an instrument such as a laser or the like is also required.

FIGS. 1C and 1D are views showing exemplary bio-bonding detecting apparatus using capacitance devices. Specifically, FIG. 1C illustrates the bio-bonding detecting apparatus using a trench type capacitance device, and FIG. 1D illustrates the bio-bonding detecting apparatus using a planar type capacitance device.

In case of using a change of characteristics of the capacitance device, there is a disadvantage in that it is difficult to form a compact size capacitance device. Since capacitance is proportional to a cross-section area and is inversely proportional to a thickness, the capacitance device is difficult to form because to facilitate a bio processing, the cross-sectional area must be increased. In the bio-bonding detecting apparatus using the trench type capacitor like in FIG. 1C, a trench is deeply formed so as to make the capacitor thinner to enlarge the cross-section area thereof. However, there is a disadvantage in that the gap, or trench, is in fact very small, making implementing the bio processing difficult. The bio-bonding detecting apparatus of FIG. 1D using a capacitor similar to that which is formed as a comb shape in a plane, also has a disadvantage in that a thickness of a metallic thin film is so small that many capacitance devices cannot easily be formed on the metallic thin film. This disadvantage results in poor sensitivity in detecting the bio-bonding.

FIG. 1E illustrates an exemplary bio-bonding detecting apparatus using light reflection. Such bio-bonding detecting apparatus using light reflection determines whether the bio-bonding has been conducted by forming probe molecules on a gold film. Light introduced thereto irradiates to detect a change of light characteristics before and after the bio-bonding. That is, after comparing a light intensity, a light wavelength and the like before and after the bio-bonding, if there is a difference, it is determined that the bio-bonding has been conducted. However, the bio-bonding detecting apparatus using the light reflection has a disadvantage in that is includes complicated construction.

### SUMMARY OF THE INVENTION

An apparatus for detecting a bio-bonding and a method thereof having a good detection efficiency, include probe biomolecules formed on a metallic thin film having an increased surface area and detecting a change of an electrical property before and after bio-bonding to determine whether the bio-bonding has been achieved.

In an exemplary embodiment, an apparatus for detecting a bio-bonding includes a signal transducer section having a substrate, a metallic thin film stepped on a surface of a certain region of the substrate, probe biomolecules formed on the metallic thin film, an input port connected with one side of the signal transducer section and configured to receive a signal for monitoring an electrical property of the signal transducer section and a detector section for characteristic quantity connected with the other side of the signal transducer section configured to monitor an electrical property of the signal transducer section.

In another exemplary embodiment, the signal transducer section may include a cap coupled to the substrate for covering an upper portion of the substrate.

In another exemplary embodiment, the signal transducer section may include a hole configured to allow a sample to be injected therethrough.

In another exemplary embodiment, the metallic thin film may be made from gold (Au) or copper (Cu). The metallic thin film may also be formed at its surface with at least one of a stud, a ball, a groove and unevenness (convex or concave).

In another exemplary embodiment, the electrical property may include impedance, current or voltage of the signal transducer section. The detector section may include one of an impedance detector, a current detector and a voltage detector.

In another exemplary embodiment, the apparatus for detecting a bio-bonding further includes an output port connected with the detector section and a second side of the signal transducer. The output port is configured to output the signal.

In another exemplary embodiment a method for detecting a bio-bonding is provided, the method including forming a signal transducer section comprising forming a metallic thin film on a surface of a certain region of an upper portion of a substrate and forming probe biomolecules on the metallic thin film, introducing a sample to the metallic thin film, monitoring a first electrical property of the signal transducer section, reacting the sample with the probe biomolecules thereby bonding them to each other, monitoring a second electrical property of the signal transducer section after the bonding reaction and comparing the first electrical property and the second electrical property to determine whether the bio-bonding has been conducted.

In another exemplary embodiment, the metallic thin film includes a stepped surface.

In another exemplary embodiment, introducing the sample includes injecting the sample through a hold in a cap, the cap being coupled to the substrate. Introducing the sample may also include injecting the sample through the metallic thin film.

In another exemplary embodiment, the reacting comprises removing extra biomolecules of the sample not bonded to the probe biomolecules. In another exemplary embodiment, the biomolecules of the sample bonded to the probe biomolecules may also be removed.

In another exemplary embodiment, the comparing the first and second electrical properties includes detection of the bio-bonding when the first and second electrical properties are different.

### BRIEF DESCRIPTION OF THE DRAWINGS

The above aspects and features of the present invention will be more apparent by describing certain embodiments of the present invention with reference to the accompanying drawings, in which:

FIGS. 1A to 1E are views to illustrate bio-bonding detecting methods and apparatuses of the prior art;

FIGS. 2A to 2C are views of exemplary embodiments of a bio-bonding detecting apparatus according to the present invention;

FIG. 3 is a flowchart of an exemplary embodiment of a method for detecting a bio-bonding according to the present invention;

FIGS. 4A to 4C are cross-sectional views of exemplary embodiments of a method for detecting a bio-bonding according to the present invention; and

FIGS. 5A to 5C are cross-sectional views of other exemplary embodiments of a method for detecting a bio-bonding according to the present invention.

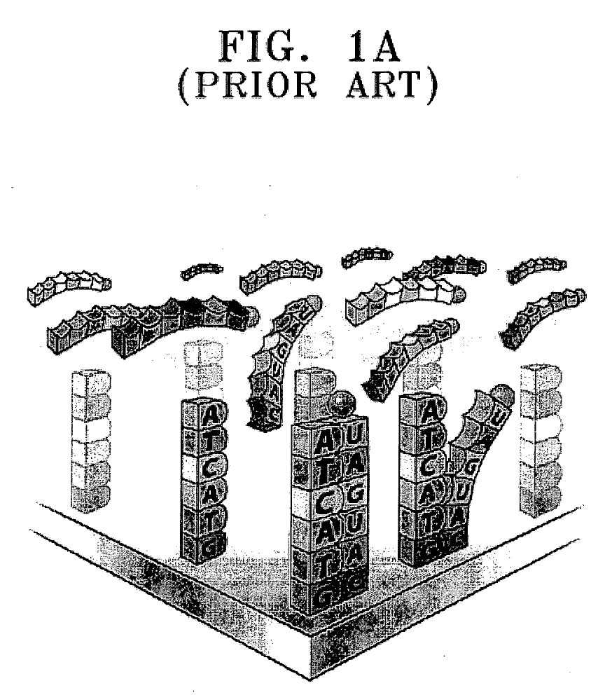

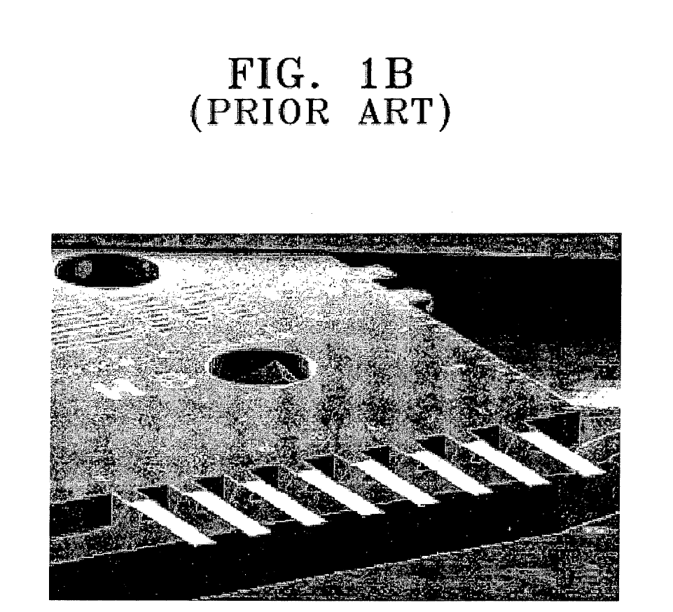

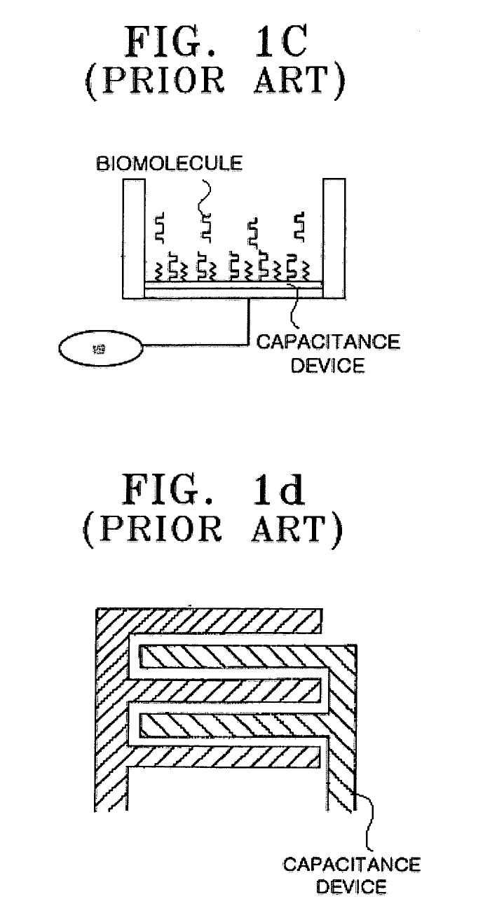

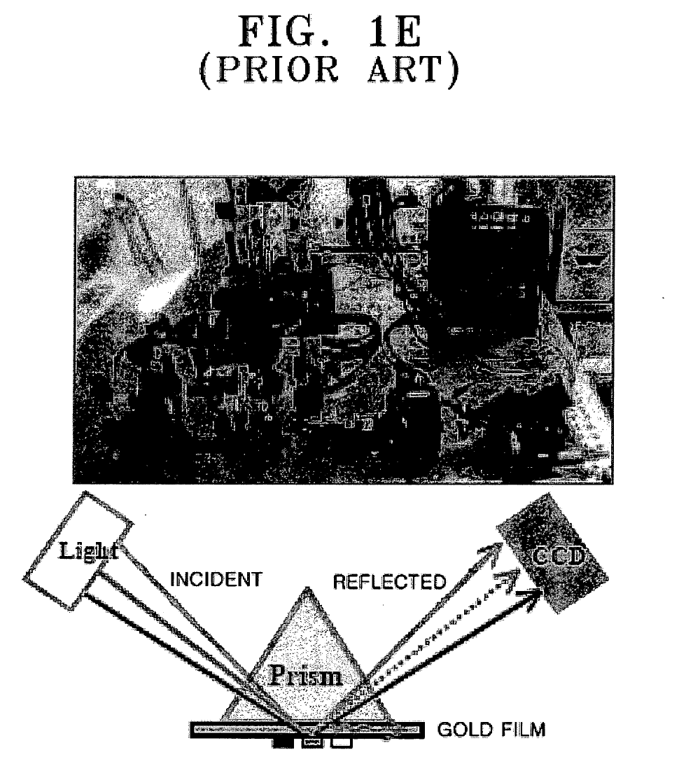

### DETAILED DESCRIPTION OF THE INVENTION

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that when an element or layer is referred to as being “on”, “connected to” or “coupled to” another element or layer, the element or layer can be directly on, connected or coupled to another element or layer or intervening elements or layers. In contrast, when an element is referred to as being “directly on,” “directly connected to” or “directly coupled to” another element or layer, there are no intervening elements or layers present. Like numbers refer to like elements throughout. As used herein, the term “and/or” includes any and all combinations of one or more of the associated listed items.

Spatially relative terms, such as “beneath”, “below”, “lower”, “above”, “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of the invention.

Hereinafter, the present invention will be described in detail with reference to the drawings.

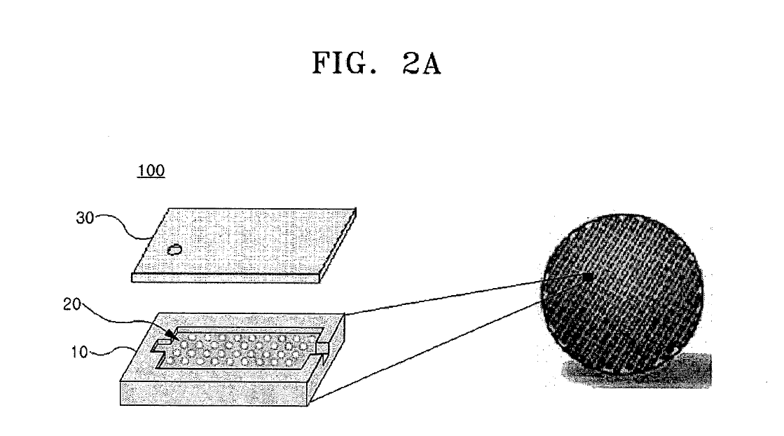

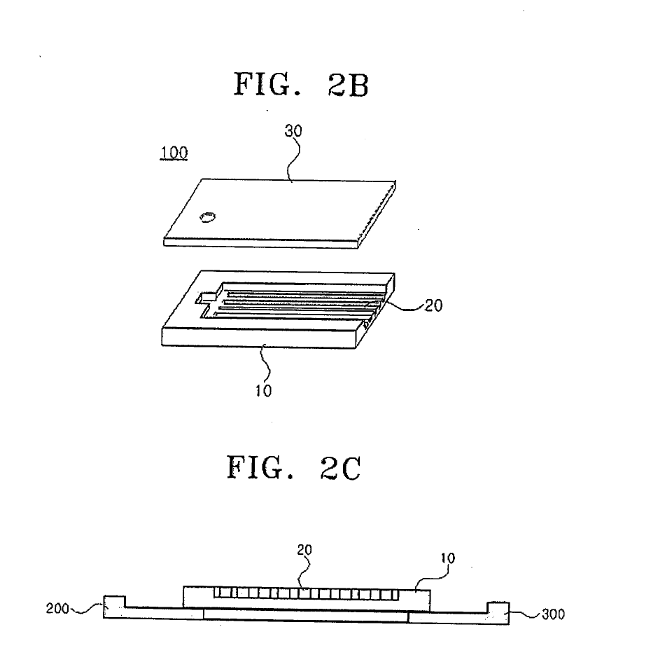

FIGS. 2A to 2C are views of exemplary embodiments of a bio-bonding detecting apparatus according to the present invention, in which FIGS. 2A and 2B illustrate a signal transducer section 100 having a substrate 10 and a metallic thin film 20 on the substrate 10. The metallic thin film 20 may have a stud, a ball, or a groove formed thereon. FIG. 2C illustrates a signal transducer section 100 in a cross-sectional view, which has an input port 200 and an output port 300 thereon.

Referring to FIGS. 2A and 2B, the signal transducer section 100 includes a substrate 10, a metallic thin film 20, a cap 30 and probe molecules (not shown). The substrate 10 and the cap 30 may be formed in a wafer level.

The metallic thin film 20 is formed to a certain region on an upper portion of the substrate 10. The metallic thin film 20 formed on the substrate 10 may include, but is not limited to, Au, Cu, or the like, as well as including any combination of the foregoing.

The metallic thin film 20, as shown in FIG. 2A, is formed such that a planar metallic thin film 20 is formed on the substrate 10. A stud or a ball may be formed on the metallic thin film 20. In exemplary embodiments, the metallic thin film 20 may be formed on the substrate 10 with grooves as shown in FIG. 2B. In alternative embodiments, the metallic thin film 20 may be of a shape of unevenness and formed on the substrate 10.

The metallic thin film 20 having a stepped surface formed with a stud, a ball or a groove may increase a surface area of the substrate 10. The surface area may be increased in order to form a large quantity of the probe biomolecules on the metallic thin film 20. The probe biomolecules can search specific information of sample biomolecules to be analyzed for immobilization of the probe biomolecules. The immobilization of a large quantity of probe biomolecules on the metallic thin film 20 with the increased surface area makes it possible to increase a detection sensitivity of the bio-bonding.

Referring to FIGS. 2A and 2C, the cap 30 is coupled to the substrate 10, so as to essentially cover the metallic thin film 20. In alternative embodiments a hole may be formed in the cap 30. The sample biomolecules to be analyzed are injected into the hole so as to be bonded to the probe biomolecules formed on the metallic thin film 20. In alternative embodiments, without forming the cap 30 on the substrate 10, the sample biomolecules may be directly injected to the upper portion of the opened metallic thin film 20 so as to be bonded to the probe biomolecules formed on the metallic thin film 20. The signal transducer section 100 may include the substrate 10, the metallic thin film 20, and the probe biomolecules (not shown) immobilized on the metallic thin film 20.

Referring to FIG. 2C, the substrate 10 and the metallic thin film 20 of the signal transducer section 100 may be formed on the input port 200 and the output port 300. The input port 200 may be connected with one side of the signal transducer section 100. The signal for monitoring an electrical property of the signal transducer section 100 is input to the input port 200. The electrical property may include, but is not limited to, impedance, current, voltage and the like, of the signal transducer section 100.

The output port 300 may be connected with the other side of the signal transducer section 100 and also with a detector section (not shown) for characteristic quantity. The detector section for characteristic quantity connected with the output port 300 may include, but is not limited to, an impedance detector, a current detector, a voltage detector or the like, according to the electrical property of the signal transducer section 100. The output port 300 is a means through which the signal input from the input port 200 may be output. The electrical properties of current, voltage, impedance and the like, that are output through the output port 300 are different according to whether or not bio-bonding is achieved of the sample biomolecules with the probe biomolecules that may be formed on the metallic thin film 200 of the signal transducer section 100.

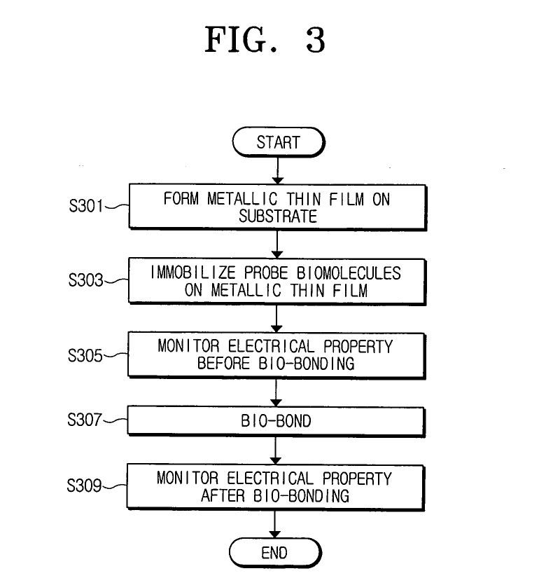

FIG. 3 is a flowchart of an exemplary embodiment of a method for detecting a bio-bonding according to the present invention.

Referring to FIG. 3, the metallic thin film 20 is formed on a certain region on the substrate 10 (S301). The metallic thin film 20 formed on the substrate 10 may include, but is not limited to, Au, Cu, or the like as well as any combination including at least one of the foregoing. In exemplary embodiments, the metallic thin film 20 may be Au because it is easily formed in a film type on the substrate 10. Additionally, the probe biomolecules may be easily immobilized on the metallic thin film 20.

The metallic thin film 20 may be formed on the substrate 10 by plating, a bumping, a deposition or any similar method suitable for the purpose described herein. The shape of the metallic thin film 20, as shown in FIG. 2A, may be formed such that a stud or a ball is included thereon. As shown in FIG. 2B, the metallic thin film 20 may be of a shape that grooves are formed thereon. In alternative embodiments, the metallic thin film 20 may be formed in any shapes having an increased surface area through the formation of a step on the surface such as an unevenness surface.

In exemplary embodiments the surface area may be increased to increase a detection sensitivity of the bio-bonding. With the increased surface area, a large quantity of the probe biomolecules may be formed on the metallic thin film 20. The probe biomolecules searching for specific information of the sample biomolecules to be analyzed are immobilized.

Next, the probe biomolecules are immobilized on the metallic thin film 20 (S303). The probe biomolecules for searching for information on the sample biomolecules to be analyzed are immobilized on the metallic thin film 20. The probe biomolecules may include, but are not limited to, DNA, RNA, protein, bimolecular, or the like. The metallic thin film 20 having its surface stepped is formed on the substrate 10 so that a large quantity of probe biomolecules may be immobilized on the metallic thin film 20 due to the increased surface area.

In exemplary embodiments, if the signal transducer section 100 includes a cap 30, after the probe biomolecules have been immobilized on the metallic thin film 20 (S303), the cap 30 having the hole is coupled to the substrate 10 so as to cover the upper portion thereof. The sample biomolecules are injected through the hole of the cap 30. In alternative embodiments, if the cap 30 is not coupled to the substrate 10, the sample biomolecules are injected through the exposed metallic thin film 20.

The electrical property of the signal transducer section 100 before the bio-bonding is monitored (S305). Before the sample biomolecules are injected to the metallic thin film 20 with the probe biomolecules formed thereon, a signal may be input to the input port 200 connected with one side of the signal transducer section 100. The signal output from the output port 300 is monitored at the detector section for characteristic quantity. The electrical property may include, but is not limited to, impedance, current, voltage and the like, of the signal transducer section 100. The detector section for characteristic quantity may include, but is not limited to, an impedance detector, a current detector, a voltage detector, or the like. If the cap 30 is coupled to the upper portion of the substrate 10, the electrical property of the signal transducer section 100 is monitored after the coupling of the cap 30 to the substrate 10.

The sample biomolecules are bio-bonded to the probe biomolecules formed on the metallic thin film 20 (S307). Where the cap 30 with the hole formed thereto is coupled to the substrate 10, the sample biomolecules are injected through the hole. Otherwise, when the cap 30 is not provided, the sample biomolecules are directly injected to the exposed metallic thin film 20. The sample biomolecules injected are bio-bonded with the probe biomolecules immobilized on the metallic thin film 20 (S307).

If the probe biomolecules are of the same shape as that of the sample biomolecules injected, the sample biomolecules are bonded to the probe biomolecules, forming the bio-bonding. Sample biomolecules not bio-bonded to the probe biomolecules are removed. The sample biomolecules bonded to the probe biomolecules may also be removed.

The electrical property of the signal transducer section 100, after the bio-bonding, is monitored (S309). The electrical property may be, but is not limited to, impedance, current and voltage. The electrical property of the signal transducer section 100 after the bio-bonding is obtained by monitoring the signal output to the output port 300 at the detector section for characteristic quantity. If the monitored value for the signal output from the output port 300 after the bio-bonding is different from that monitored by the detector section for characteristic quantity before the bio-bonding, it is determined that the bio-bonding has been conducted. The value difference between the electrical property from before and after the bio-bonding may be detected. Whether bio-bonding has occurred and a quantified amount of the bio-bonding may also be detected.

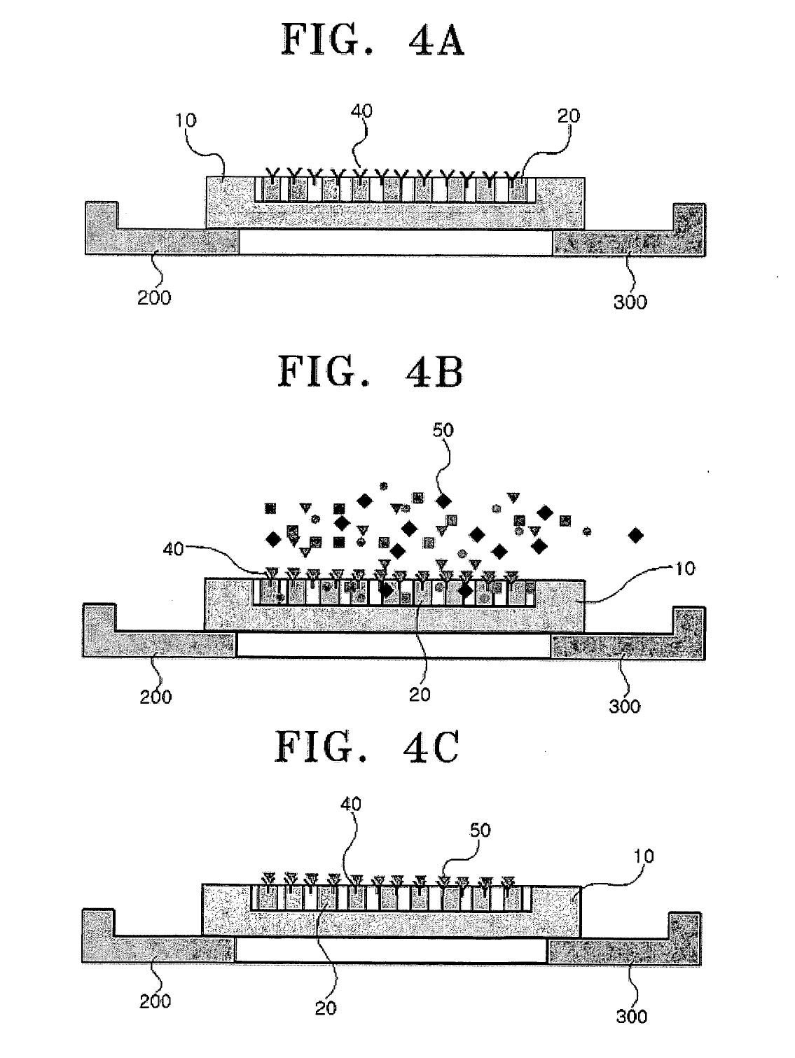

FIGS. 4A to 4C are cross-sectional views of exemplary embodiments of a method for detecting a bio-bonding according to the present invention. FIGS. 4A to 4C illustrate a bio-bonding detecting apparatus in which a metallic thin film 20 having grooves is formed on a substrate 10, and a cap 30 is not provided on the substrate 10. FIG. 4A shows the bio-bonding apparatus in a state before the bio-bonding, FIG. 4B shows the bio-bonding apparatus in a state of being bio-bonded, and FIG. 4C shows the bio-bonding apparatus in a state after the bio-bonding.

Referring to FIG. 4A, the probe biomolecules 40 are immobilized on the metallic thin film 20 on the substrate 10. In order to detect whether the bio-bonding is conducted, the electrical property of the signal transducer section 100 before the bio-bonding is detected.

Referring to FIG. 4B, the sample biomolecules 50 to be analyzed are injected so as to bio-bond to the probe biomolecules 40 immobilized on the metallic thin film 20. If the probe biomolecules 40 are of the same shape as that of the sample biomolecules 50, the sample biomolecules 50 are bio-bonded to the probe biomolecules 40 immobilized on the metallic thin film 20.

Referring to FIG. 4C, after the bio-bonding, excess sample biomolecules 50 not bio-bonded to the probe biomolecules 40 and the sample biomolecules 50 that are bonded to the probe biomolecules 40 may be removed. The electrical property of the signal transducer section 100 after the bio-bonding is detected. If the monitored electrical property of the signal transducer section 100 after the bio-bonding is different from that before the bio-bonding, it is determined that the bio-bonding has been conducted.

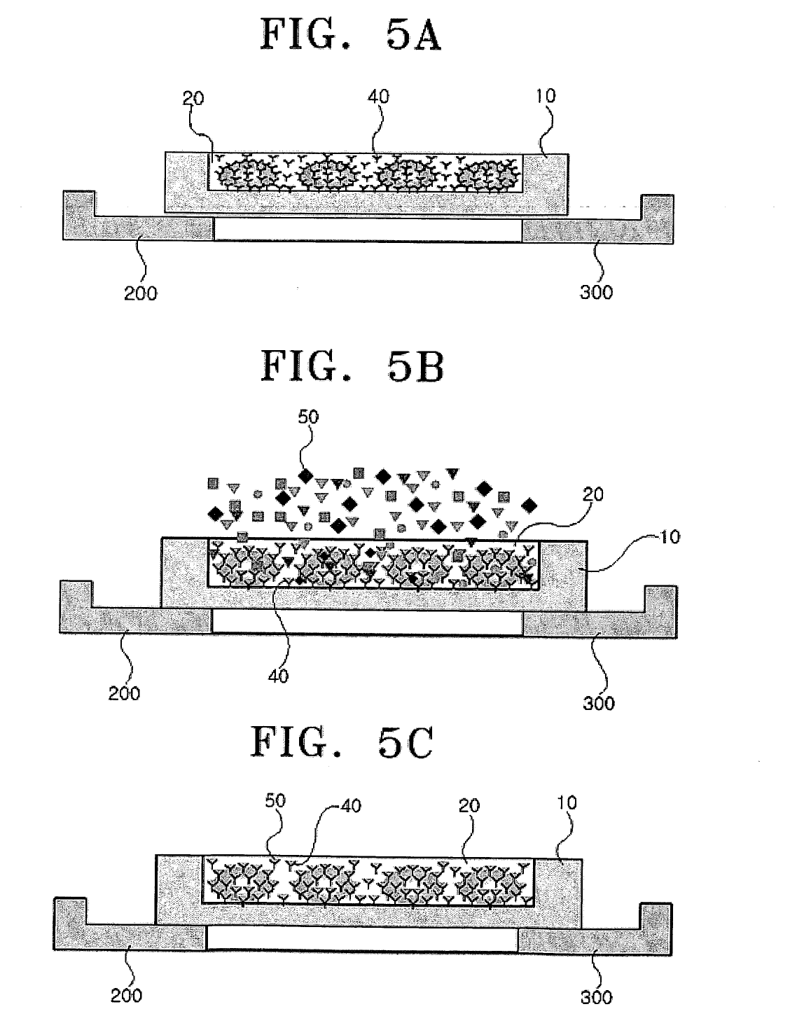

FIGS. 5A to SC are cross-sectional views of other exemplary embodiments of a method for detecting a bio-bonding according to the present invention. FIGS. SA to SC illustrate a bio-bonding detecting apparatus in which a metallic thin film 20, having a stud or a ball, for example, is formed on a substrate 10. FIG. 5A shows the bio-bonding apparatus in a state before the bio-bonding, FIG. 5B shows the bio-bonding apparatus in a state of being bio-bonded, and FIG. SC shows the bio-bonding apparatus in a state after the bio-bonding. The metallic thin film 20 formed on the substrate 10 in FIGS. SA to SC has a difference only in its shape compared to metallic thin film 20 formed on the substrate 10 in FIGS. 4A to 4C, but the method for detecting the bio-bonding is the same as described regarding FIGS. 4A to 4C.

According to the present invention, the probe biomolecules are immobilized on the metallic thin film having an increased surface area. Bio-bonding is detected through the monitoring the electrical property of the signal transducer section before and after the bio-bonding. Advantageously, a detection sensitivity is increased, a structure of the bio-bonding detecting apparatus is simplified, and a high-priced reader is not required.

Furthermore, the bio-bonding may be detected when the signal transducer section includes a wafer level configuration and during a packaging process, such that the detecting of the bio-bonding may be conducted when the signal transducer section includes a wafer level configuration. Advantageously, a compact size of the apparatus and a low working cost may be achieved.

The foregoing embodiment and advantages are merely exemplary and are not to be construed as limiting the present invention. The present teaching can be readily applied to other types of apparatuses. Also, the description of the embodiments of the present invention is intended to be illustrative, and not to limit the scope of the claims, and many alternatives, modifications, and variations will be apparent to those skilled in the art.

What is claimed is:

1. An apparatus for detecting a bio-bonding, comprising:
a signal transducer section comprising a substrate, a metallic thin film formed on a surface of a certain region of the substrate, wherein the metallic thin film is stepped, and probe biomolecules formed on the metallic thin film;
an input port connected with a first side of the signal transducer section and configured to receive a signal for monitoring an electrical property of the signal transducer section; and
a detector section for characteristic quantity connected with a second side of the signal transducer section configured to monitor the electrical property of the signal transducer section.

2. The apparatus as claimed in claim 1, wherein the signal transducer section further comprises a cap coupled to the substrate.

3. The apparatus as claimed in claim 2, wherein the cap comprises a hole configured to allow a sample to be injected therethrough.

4. The apparatus as claimed in claim 1, wherein the metallic thin film comprises gold (Au) or copper (Cu).

5. The apparatus as claimed in claim 1, wherein the metallic thin film comprises a stud, a ball, a groove, unevenness on its surface, or any combination including at least one of the foregoing.

6. The apparatus as claimed in claim 1, wherein the electrical property comprises impedance, current, voltage, or any combination including at least one of the foregoing.

7. The apparatus as claimed in claim 1, wherein the detector section for characteristic quantity comprises an impedance detector, a current detector, a voltage detector, or any combination including at least one of the foregoing.

8. The apparatus as claimed in claim 1, wherein the probe biomolecules are immobilized on the metallic thin film.

9. The apparatus as claimed in claim 1, wherein the probe biomolecules comprise DNA, RNA, protein, or any combination including at least one of the foregoing.

10. The apparatus as claimed in claim 1, further comprising an output port connected with the detector section and a second side of the signal transducer, wherein the output port is configured to output the signal.

11. A method for detecting a bio-bonding, the method comprising:
forming a signal transducer section comprising forming a metallic thin film on a surface of a certain region of a substrate and immobilizing probe biomolecules on the metallic thin film;
introducing a sample to the metallic thin film;
monitoring a first electrical property of the signal transducer section;
reacting the sample with the probe biomolecules, thereby bonding the sample and the probe biomolecules to each other;
monitoring a second electrical property of the signal transducer section after the reacting; and
comparing the second electrical property with the first electrical property to detect whether the bio-bonding has been conducted.

12. The method as claimed in claim 11, wherein the metallic thin film comprises a stepped surface.

13. The method as claimed in claim 11, wherein the forming of the metallic thin film comprises plating, a bumping, a deposition, or any combination including at least one of the foregoing.

14. The method as claimed in claim 11, wherein the introducing a sample comprises injecting the sample through the metallic thin film.

15. The method as claimed in claim 11, wherein the introducing a sample comprises injecting the sample through a hole in a cap, the cap being coupled to the substrate.

16. The method as claimed in claim 11, wherein the reacting comprises removing excess parts of the sample not bonded to the probe biomolecules.

17. The method as claimed in claim 11, wherein the reacting comprises removing parts of the sample bonded to the probe biomolecules.

18. The method as claimed in claim 11, wherein the comparing comprises the detection of the bio-bonding when the first and second electrical properties are different.

19. The method as claimed in claim 11, wherein the monitoring is performed by a detector section.

20. An apparatus for detecting a bio-bonding, comprising:
a signal transducer section comprising a substrate, a metallic thin film formed on a surface of a certain region of the substrate, wherein the metallic thin film is stepped, and probe biomolecules formed on the metallic thin film;
an input port connected with a first side of the signal transducer section and configured to receive a signal for monitoring an electrical property of the signal transducer section; and
an output port connected with a second side of the signal transducer, wherein the output port is configured to output the signal.
